(19) United States
(12) Patent Application Publication
(10) Pub. No.: US 2020/0039907 A1
(43) Pub. Date: Feb. 6, 2020

(54) Novel method for extraction of Oxyresveratrol from Artocarpus hirsutus

(71) Applicants: Muhammed MAJEED, East Windsor, NJ (US); Kalyanam NAGABHUSHANAM, East Windsor, NJ (US); Mahadeva NAYAK, Bangalore (IN); Nagarajan ANANTHANARAYANAN, Bangalore (IN)

(72) Inventors: Muhammed Majeed, Edison, NJ (US); Kalyanam Nagabhushanam, East Windsor, NJ (US); Mahadeva Nayak, Bangalore (IN); Nagarajan Ananthanarayanan, Bangalore (IN)

(21) Appl. No.: 16/471,148

(22) PCT Filed: Dec. 22, 2016
(86) PCT No.: PCT/US16/68161

§ 371 Date: Jun. 19, 2019

(51) Int. Cl. C07C 37/68; C07C 39/215

(52) U.S. Cl. CPC C07C 37/685; C07C 39/215

(57) ABSTRACT

Disclosed is a novel method for extraction of Oxyresveratrol from Artocarpus hirsutus.

### CROSS-REFERENCE TO RELATED PATENT APPLICATIONS

This is National Phase Application of PCT application no. PCT/US 16/68161 filed on 22 Dec. 2016.

### FIELD OF THE INVENTION

The present invention relates to a method for extraction of Oxyresveratrol from Artocarpus hirsutus (A. hirsutus).

### BACKGROUND OF THE INVENTION

### Description of Prior Art

Oxyresveratrol is a hydroxyl-substituted stilbene found in the roots, leaves, stem and fruit of many widely distributed plants including Moraceae, Liliaceae, Gnetaceae, etc. The biological activity of this compound is well documented in the following scientific articles;
1. Li Xu, Chao Liu, Wei Xiang, Hu Chen, Xiaoli Qin and Xianzhi Huang, Advances in the Study of Oxyresveratrol, International Journal of Pharmacology, 2014, 10, 44-54
2. Likhitwitayawuid, K., Somsute, A., Sritularak, B., Ploypradith, P., Chemical transformations of oxyresveratrol (trans-2,4,3′,5′-tetrahydroxystilbene) into a potent tyrosinase inhibitor and a strong cytotoxic agent. (Bioorganic & Medicinal Chemistry Letters, 2006, 16, 5650-5653)

It is also reported for its modest inhibition of HIV (Likhitwitayawuid K, Sritularak B, Benchanak K, Lipipun V, Mathew J, Schinazi R F, Phenolics with antiviral activity from Millettia erythrocalys and Artocarpus lakoocha, Nat Prod Res. 2005 February; 19(2):177-82).

Oxyresveratrol has been isolated from several Artocarpus species (A. lakoocha Roxb.; A. champlasha Roxb.; A. champlasha Roxb.; A. heterophyllus; A. gomezianus Wall.). (Nasapon Povichit, Ampai Phrutivorapongkul, Maitree Suttajit and Pimporn Leelapornpisid, Antiglycation and antioxidant activities of Oxyresveratrol extracted from the heartwood of Artocarpus lakoocha Roxb., Maejo Int. J. Sci. Technol. 2010, 4(03), 454-461) using chromatographic techniques like HPLC, Column chromatography etc. While these techniques are expensive and time consuming, a simple inexpensive and reliable method of isolation is technically desired for the large scale industrial production of Oxyresveratrol. The present invention solves such technical problems by disclosing a simple solvent-solvent purification method of Oxyresveratrol from A. hirsutus with high recovery.

It is the principle objective of the invention to disclose a simple, commercially viable method for the extraction of Oxyresveratrol from Artocarpus hirsutus.

The present invention fulfils the aforesaid objectives and provides further related advantages.

### SUMMARY OF THE INVENTION

Disclosed is method for extraction of Oxyresveratrol from A. hirsutus. More specifically, the invention discloses a novel method for extraction of Oxyresveratrol from A. hirsutus using a simple solvent-solvent purification method.

### BRIEF DESCRIPTION OF THE DRAWINGS (FIGS. 1 and 2)

FIG. 1 shows ¹H-NMR (DMSO-d₆, 300 MHz): δ 9.72 (1H, s, 5-OH), 9.54 (1H, s, OH), 9.32 (2H, s, OH), 7.33 (1H, d, J=8.7 Hz, H-6), 7.14 (1H, d, J=16.5 Hz, H-α), 6.76 (1H, d, J=16.5 Hz, H-β), 6.34 (2H, d, J=2.1 Hz, H-2′ and H-6′), 6.30 (1H, d, J=2.4 Hz, H-3), 6.24 (1H, dd, J=8.4 and 2.1 Hz, H-5), 6.06 (1H, t, J=2.1 Hz, H-4′).

FIG. 2 shows ¹³C NMR of Oxyresveratrol (DMSO-d₆, 75 MHz): δ 158.75 (C-3′ and 5′), 158.37 (C-4), 156.30 (C-2), 140.36 (C-1′), 127.60 (C-6), 124.97 (C-β), 123.57 (C-α), 115.61 (C-1), 107.60 (C-5), 104.34 (C-2′ and 6′), 102.87 (C-4′), 101.70 (C-3).

FIGS. 3a and 3b shows Liquid Chromatography Mass Spectrometry (LCMS) of compound Oxyresveratrol obtained from A. hirsutus

FIG. 4 shows High Performance Liquid Chromatography (HPLC) of compound Oxyresveratrol obtained from A. hirsutus

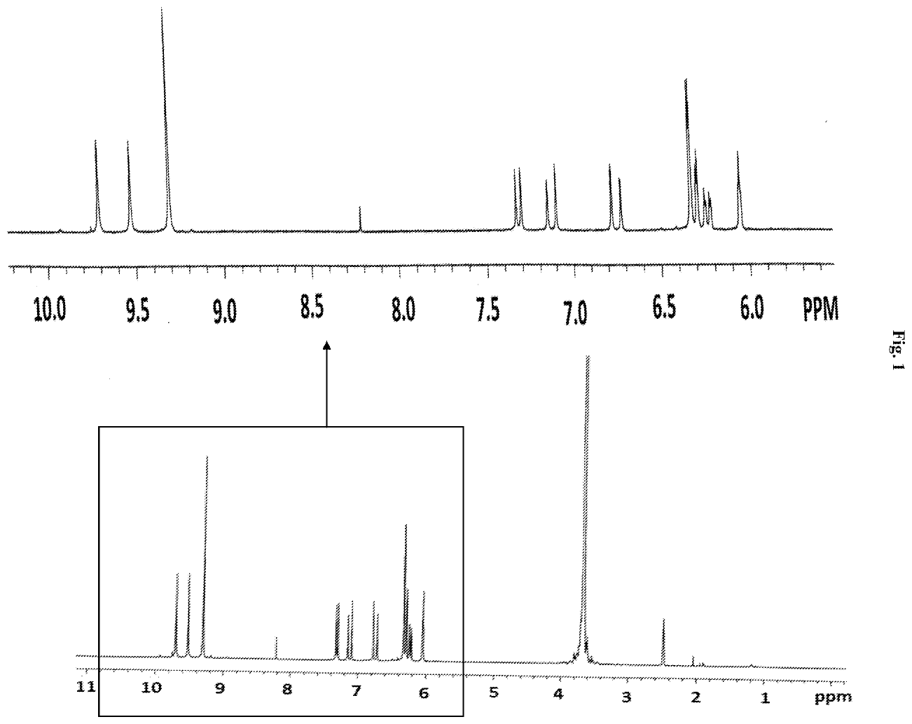

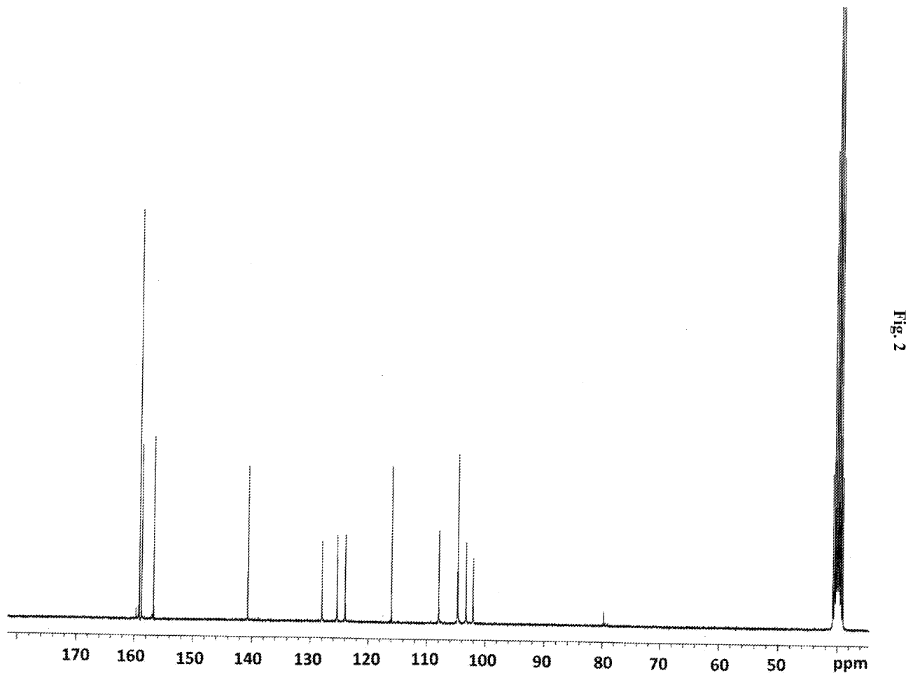

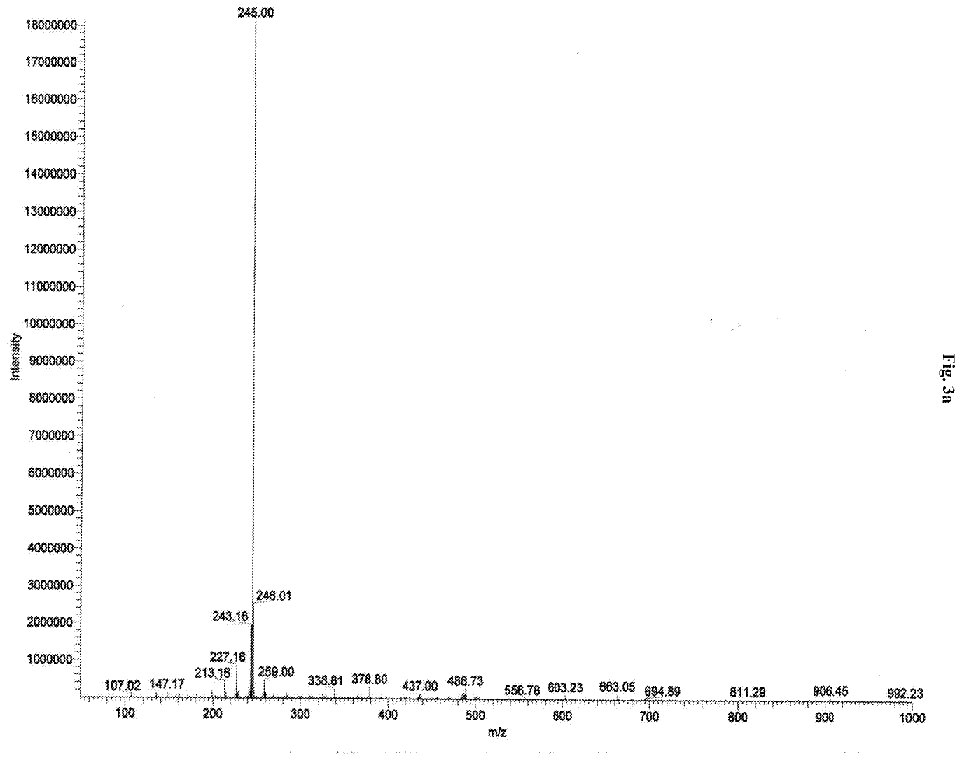

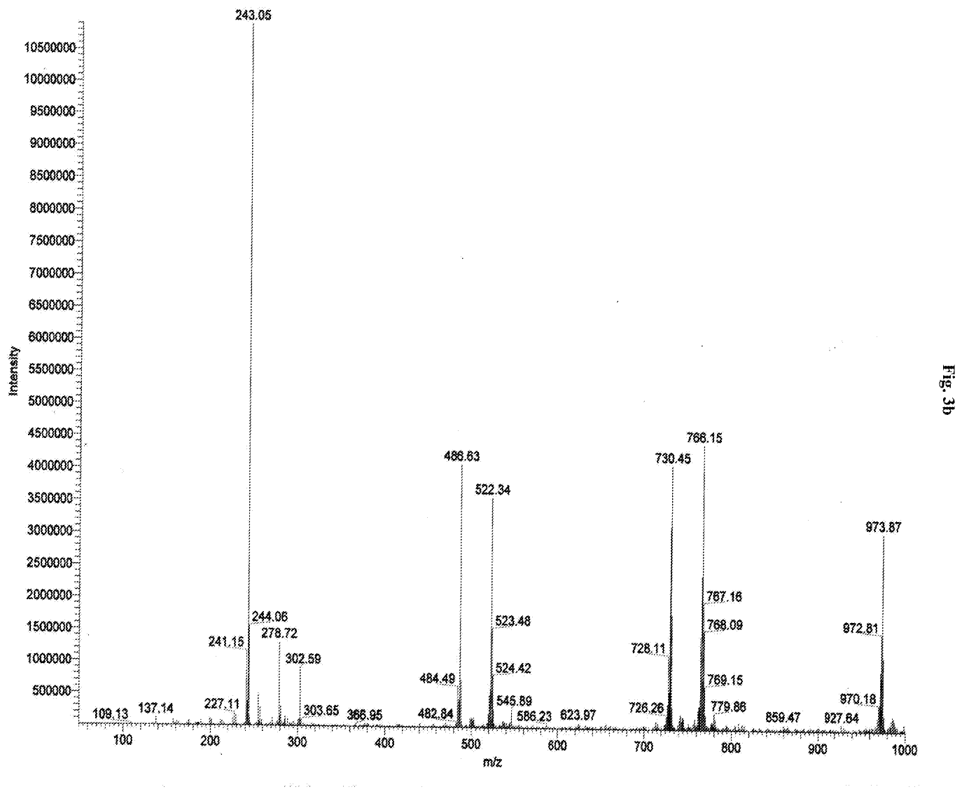

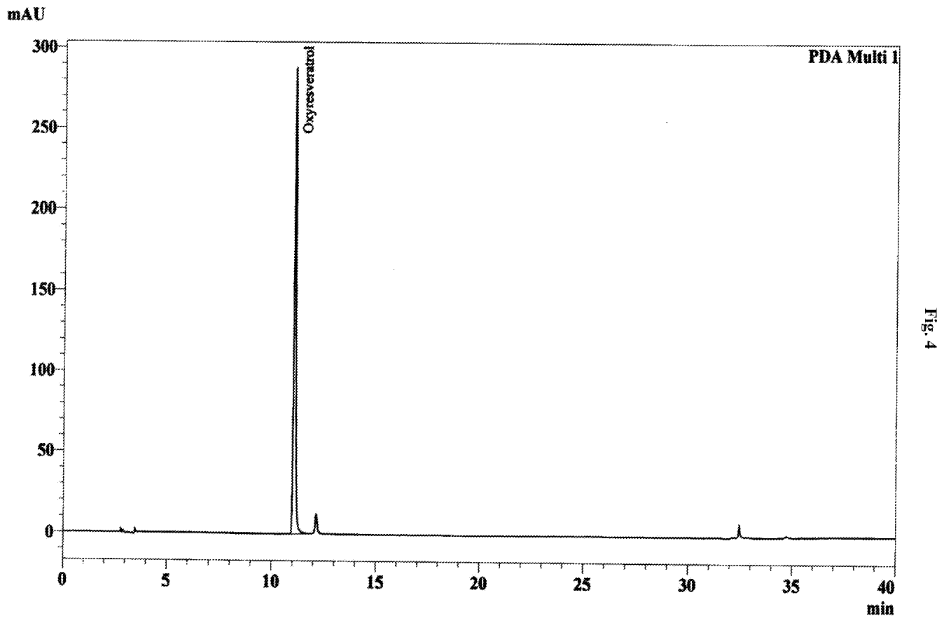

### DETAILED DESCRIPTION OF THE MOST PRFFRED EMBODIMENT

In the most preferred embodiment, the present invention discloses a novel method for extraction of Oxyresveratrol from Artocarpus hirsutus, said method comprising steps of:
a) Cutting, drying the wood of A. hirsutus and pulverising to coarse powder;
b) Extracting powdered material from step a with hot ethanol (9 volumes) completely
c) Filtering to separate the ethanol extract from step b;
d) Concentrating the extract from step c under vacuum at 50-55° C. to thick paste;
e) Drying the extract from step d completely in vacuum tray dryer at 65-70° C. to get powder;
f) Dissolving the powder from step e in ethanol (2 volumes);
g) Addition of the ethanolic extract from step f slowly into 10 volumes of water under continuous stirring;
h) Separating the insolubles obtained from step g to get clear layer by filtration;
i) Washing the clear layer from step h with chloroform (1 volume) and discard the chloroform layer
j) Extracting the clear layer from step i with ethyl acetate (1 volume)
k) Concentrating the ethyl acetate layer from step j under vacuum at 50-55° C. to thick paste;
l) Drying the concentrated extract from step k to get powder in vacuum tray dryer at 65-70° C.;
m) Dissolving the powder from step 1 in water (4 volumes) and heating upto 80-90° C. for 8 h under stirring;
n) Cool the water layer from step in to 10-15° C. for 8 h and allowed for crystallization;
o) Filtering the crystals obtained from step n;
p) Drying the crystals from step o in a vacuum at 70-75° C.;
Characterizing the crystals from step p as Oxyresveratrol from its ¹H and ¹³C NMR spectra as well as from its LCMS spectrum as represented in STR#1

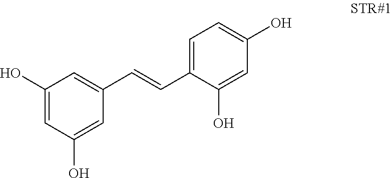 [SMILES: Oc1cc(O)cc(/C=C/c2ccc(O)cc2O)c1]

Other features and advantages of the present invention will become apparent from the following more detailed description, taken in conjunction with the accompanying images, which illustrate, by way of example, the principle of the invention.

### EXAMPLE

Preparation of the Extract

Artocarpus hirsutus wood was collected and cut into small pieces, dried in shade. The dried material was pulverised to a coarse powder and stored in an air tight container. The powdered material was extracted completely with soaked volume of hot ethanol (3 volumes×3 times). The ethanolic extracts were separated by filtration and collected in a clean container. The combined extracts were concentrated to thick paste under vacuum at 50-55° C. and dried in vacuum tray drier to get powder. The powdered extract was collected and stored at room temperature in air tight container.

Isolation and Characterization of Active Compound

The powdered ethanolic extract from A. hirsutus wood was redissolved in a small amount (2 volumes) of ethanol and poured into 10 volumes of water. The material was filtered to separate the water soluble and water insoluble fraction. The water soluble fraction was then extracted with ethyl acetate (1 volume) and the ethyl acetate layer was dried completely to get the powder. The powder obtained from ethyl acetate fraction was then poured into water (4 volumes) and stirred at 80-90° C. for 8 h. After cooling at room temperature, the solution was filtered and the crystallised solid material was dried under vacuum at 65 -70° C. This filtered crystallised material was characterized as Oxyresveratrol from its ¹H and ¹³C NMR spectra as well as from its LCMS spectrum and was comparable with the reported values. The isolated Oxyresveratrol shows the following values: C₁₄H₁₂O₄. m. p.: 191-194° C. Color: pale brown fine powder, APCI-MS m/z 245.00 (M+H⁺) and 245.05 (M-H⁻) (C₁₄H₁₂O₄ requires 244.2426). Purity of the compound was further verified by HPLC.

While the invention has been described with reference to a preferred embodiment, it is to be clearly understood by those skilled in the art that the invention is not limited thereto. Rather, the scope of the invention is to be interpreted only in conjunction with the appended claims.

We claim;:

1. A novel method for extraction of Oxyresveratrol from Artocarpus hirsutus, said method comprising steps of:
a) Cutting, drying the wood of A. hirsutus and pulverising to coarse powder;
b) Extracting powdered material from step a with hot ethanol (9 volumes) completely
c) Filtering to separate the ethanol extract from step b;
d) Concentrating the extract from step c under vacuum at 50-55° C. to thick paste;
e) Drying the extract from step d completely in vacuum tray dryer at 65-70° C. to get powder;
f) Dissolving the powder from step e in ethanol (2 volumes);
g) Addition of the ethanolic extract from step f slowly into 10 volumes of water under continuous stirring;
h) Separating the insolubles obtained from step g to get clear layer by filtration;
i) Washing the clear layer from step h with chloroform (1 volume) and discard the chloroform layer
j) Extracting the clear layer from step i with ethyl acetate (1 volume)
k) Concentrating the ethyl acetate layer from step j under vacuum at 50-55° C. to thick paste;
l) Drying the concentrated extract from step k to get powder in vacuum tray dryer at 65-70° C.;
m) Dissolving the powder from step 1 in water (4 volumes) and heating upto 80-90° C. for 8 h under stirring;
n) Cool the water layer from step m to 10-15° C. for 8 h and allowed for crystallization;
o) Filtering the crystals obtained from step n;
p) Drying the crystals from step o in a vacuum at 70-75° C.;
q) Characterizing the crystals from step p as Oxyresveratrol from its ¹H and ¹³C NMR spectra as well as from its LCMS spectrum as represented in STR#1 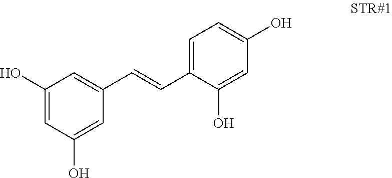 [SMILES: Oc1cc(O)cc(/C=C/c2ccc(O)cc2O)c1]
